(19) United States
(12) Patent Application Publication
(10) Pub. No.: US 2017/0231794 A1
(43) Pub. Date: Aug. 17, 2017

(54) ORTHOPAEDIC BRACE AND METHOD FOR MANUFACTURING AN ORTHOPAEDIC BRACE

(71) Applicant: Ryan Church, Toronto (CA)

(72) Inventor: Ryan Church, Toronto (CA)

(21) Appl. No.: 15/501,912

(22) PCT Filed: Aug. 11, 2015
(86) PCT No.: PCT/CA2015/050759

§ 371 Date: Feb. 6, 2017

Related U.S. Application Data
(60) Provisional application No. 62/035,779, filed on Aug. 11, 2014.

(51) Int. Cl. A61F 5/01; A61F 5/058

(52) U.S. Cl. CPC A61F 5/0111; A61F 5/0585; A61F 5/0106

(57) ABSTRACT

Various orthopaedic brace configurations and elements to be applied to the lower leg and foot are disclosed here, which include a rigid monocoque exoskeleton, foam padded interior, a plurality of retention devices, a plurality of stabilizers extendable through a spring loaded ratcheting mechanism, and a plurality of smaller stabilizer structures. This orthopaedic brace limits inversion, eversion and planterflexion of the ankle and limits flexion, extension, medial rotation and lateral rotation of the knee and hip joint.

### CROSS-REFERENCE TO RELATED APPLICATION

The present invention is related to, and claims priority from, U.S. Provisional Patent Application No. 62/035,779 with the title ‘Orthopaedic Foot Brace’ filed on 11, Aug. 2014 by Ryan Church which is herein incorporated by reference.

### FIELD OF THE INVENTION

The following relates generally to orthopaedic devices and more particularly to an orthopaedic brace that limits inversion, eversion and planterflexion of the ankle and limits flexion, extension, medial rotation and lateral rotation of the knee and hip joint. The following also relates to a method of manufacturing such a device by way of 3D scanning and 3D printing to achieve customization.

### BACKGROUND OF THE INVENTION

Orthopaedic ankle braces are generally composed of a hard exoskeleton of fibreglass impregnated resin or plastic that conforms to the ankle. This exoskeleton conforms to the ankle in the same way a ski-boot may, covering the ankle with a hard shell, while proving a padded inner surface which comes in contact with the leg, thus providing cushioning and comfort. US. Pat. Nos. 5,217,431 by Gregory Kowalczyk et al. is a boot-type brace. U.S. Pat. No. 6,406,450 B1 by Gregory Kowalczyk et al. is a brace for supporting an ankle comprising a flexible material and having a medial side portion dimensioned and configured to extend along a medial side of a user's leg and foot.

Commonly known orthopaedic ankle braces contain a unitary exoskeleton that surrounds the posterior portion of the calf and planter portion of the foot, while having straps to secure the anterior portion of the calf and superior portion of the foot. These straps are generally adjustable and allow for the accommodation of various calf and foot sizes. Furthermore, the exoskeleton travels to the mid portion of the calf or just above.

A commonly known orthopaedic ankle brace contains vent holes that allow for ventilation of the leg. This prevents perspiration of the leg and reduces healing time, while allowing for easy cleaning. On the interior of this exoskeleton, there is a liner arranged within said shell to cushion the major portion of the lower leg. U.S. Pat. No. 6,406,450 B1 by Gregory Kowalczyk et al. and U.S. Pat. No. 8,012,112 B2 by Alessandro Aldo Barberio similarly describes vent holes.

A commonly known orthopaedic ankle brace may also be used as a walking brace. Once the damaged portion of the body part has begun to heal and has stabilized, it is known that a more rapid recovery can sometimes be obtained by gradually and progressively permitting the injured body part to bear weight and undergo mild exercise with the use of an orthopaedic brace. However, attempts to combine both walking and stationary orthopaedic braces applied to the ankle region are still nascent. Thus, there may be room for invention in this field. U.S. Pat. No. 8,012,112 B2 by Alessandro Aldo Barberio describes a walking brace, however it is to provide therapeutic pressure to a person's lower leg. US 2009/0227927 A1 by Michael J. Frazer also describes a walking brace for providing therapeutic pressure to the ankle and lower leg of a person, though it comprises an exterior stirrup frame construction with a full length sole portion.

Orthopaedic hip replacement, knee replacement, or surgical intervention involving any portion of the lower limbs and requiring at least partial immobilization has seen relatively little advancement in medical devices that may aid the healing time, comfort or mobility for the patient post-operation. Medical devices and/or braces that prevent medial rotation and lateral rotation of the knee or hip joint are even more nascent, yet the prevention of these rotational forces are critical to the successful healing regime post-operation. Thus, there may be a need for such a device.

Further, the manufacturing of such a device may be done while the patient is in surgery, having had measurements taken pre-operation. Measurements that could be as exacting as possible, while being as non-invasive as possible would be desirable for both patient and healthcare practitioner, saving time and money while reducing unnecessary intrusion.

Biomimetics—the imitation of nature when addressing complex engineering problems—has gained attention in the fields of medicine and materials. However, the application of biomimetics to specific problems in the field of orthopaedic braces, such as those involving ankle, knee and hip stabilization, is still nascent. Attempts to solve complex structural problems using biomimetics without careful consideration have often failed to take into account certain key characteristics such as scale and form to functional fit. U.S. Pat. No. 6,942,628 B1 by Richard L. Watson claims a material for the formation of an orthopaedic cast. Said material is pliable and formable prior to and during application and hardenable after application, said material comprising: a mesh fabric comprising a plurality of layers of fibres defining a honeycomb skeletal array of aligned hexagonal cells passing through said plurality of layers. However, the material described is not biomimetic, nor is it in the formation of a voronoi pattern, which increases structural integrity and breathability while limiting material use maximally.

Hip abduction pillows are generally used following hip surgery when immobilization or post-operative positioning is required. These pillows are generally made from foam that forms a triangular shape and is placed in between the legs. However, this product can be hot, itchy and/or generally uncomfortable. Thus, there may be an opportunity to provide an alternative product that fulfills or exceeds the same immobilization requirements, while maximizing comfort and minimizing the material used, time spent and cost expended.

### SUMMARY OF THE INVENTION

In view of the above, an orthopaedic device, and more particularly to an orthopaedic brace that limits inversion, eversion and planterflexion of the ankle and limits flexion, extension, medial rotation and lateral rotation of the knee and hip joint through stabilizer structures associated with a rigid exoskeleton, the stabilizer structure dimensioned to interface with a generally horizontal support surface to, while being supported, inhibit rotation of the rigid exoskeleton about the major axis. This brace that is given is a rigid, generally L-shaped exoskeleton having an elongate, open-topped channel dimensioned to receive and seat a lower leg and foot of a patient, wherein the lower leg while seated within the rigid exoskeleton is aligned with a major axis of the rigid exoskeleton. This maintains the proper healing position of the hip, knee and ankle, unlike current methods of post-operative methods. The following also relates to a fully-customizable manufacturing method by way of 3D scanning and additive manufacturing that embeds within the workflow of a hospital.

According to an embodiment, there is provided an orthopaedic brace that contains a monocoque exoskeleton, foam padded interior and retention devices for securing the patients leg in said brace; the monocoque exoskeleton and foam padded interior comprising two separate parts joined together.

According to an aspect, the monocoque exoskeleton may be composed of fibreglass impregnated resin and/or any plastic or polymer suitable for extracorporeal medical devices and the interior being a foam suitable for extracorporeal medical devices such as egg-crate foam.

According to an aspect, any variation of sizes in these two separate pieces may be allowed such that a proper fit is achieved.

According to another aspect, the foam is located only where the heel makes contact with the exoskeleton when the body is supine and where the foot makes contact with the exoskeleton.

According to another embodiment, the monocoque exoskeleton and/or foam padded interior and/or retention devices may comprise a biomimetic Voronoi pattern.

According to an aspect, the monocoque exoskeleton and/or foam padded interior and/or retention devices may comprise a hexagonal-patterned structure in the range of 1-2 centimetres.

According to another embodiment, stabilizer structures associated with the rigid exoskeleton of the orthopaedic brace are dimensioned to interface with a generally horizontal support surface to, while being supported, inhibit rotation of the rigid exoskeleton about the major axis.

According to an aspect of this embodiment, at least one stabilizer plate depending from a side of the rigid exoskeleton of the orthopaedic brace opposite the open-mouth of the channel and terminating at a distal edge that runs generally transverse to the major axis. These may extend from opposite sides of the open mouth of the channel of the rigid exoskeleton.

According to another embodiment, the extendable stabilizers have an upper portion extending generally along the rigid exoskeleton and a lower portion extending generally away from the rigid exoskeleton.

According to an aspect of this embodiment, the lower portion is selectively bendable and thinner with respect to the upper portion thereby to permit selective adjustment. Each of the stabilizer legs is selectively movable and pivotable between a retracted position and a stabilizing position.

According to another embodiment, there is provided a method of achieving leg abduction through the use of the orthopaedic brace. This method replaces an existing abduction pillow which is generally placed between the legs. Due to the weight of the brace in keeping the leg in one solitary position, it may form equally well both on its own or as a tandem brace to fulfill the requirements that the abduction pillow currently fulfills.

According to another embodiment, there is provided a method of improving the stability of the hip joint after surgery using the current invention.

According to another aspect, there is provided a method for improving the quality of care of the hip joint after surgery.

According to another embodiment, there is provided a method of manufacturing an orthopaedic brace.

According to an aspect, the method comprising 3D scanning the foot, ankle and lower leg, to produce a computer representation of the inner layer of the cast, whereby this cast may then fit into the outer Voronoi cast which is pre-made.

According to an aspect, the cast made through 3D scanning and the Voronoi exoskeleton are printed together.

### BRIEF DESCRIPTION OF THE DRAWINGS

Embodiments of the invention will now be described with reference to the appended drawings in which:

FIG. 1 is a detailed side perspective of the orthopaedic brace, including exoskeleton, the retention devices, the extendable stabilizers, and an example of the smaller stabilizer structures.

FIG. 2 is an end-on view of the planter portion of the orthopaedic brace exoskeleton.

FIG. 3 is a side perspective of the extendable stabilizers with their spring loaded ratcheting mechanism.

FIG. 4 shows several examples of the Voronoi pattern which could be used, including a view of the pattern as it fills the 3D space of an elongated rectangle.

FIG. 5 is a side perspective of a retention device showing the Voronoi pattern.

FIG. 6 is a side perspective of the rigid monocoque exoskeleton showing a hexagonal-patterned structure in the range of 1-2 centimetres.

### DETAILED DESCRIPTION

Reference will now be made in detail to the various embodiments of the invention, one or more examples of which are illustrated in the figures. Each example is provided by way of explanation of the invention, and is not meant as a limitation of the invention. For example, features illustrated or described as part of one embodiment can be used on or in conjunction with other embodiments to yield yet a further embodiment. It is intended that the present invention includes such modifications and variations.

The present patent application includes description of opportunities for improving on the traditional aspects of stabilizing the ankle, knee and hip post-surgery. The present patent application yields to a novel orthopaedic foot brace assembly with a unique biologically-inspired shape that can conveniently be customized to the patient's bodily requirements and contours. Further, the following includes description of opportunities for improving on the traditional aspects of an orthopaedic foot brace so that the weight may be decreased while simultaneously increasing structural strength and allowing the brace to be used in the stabilization of the hip, knee and ankle joints in both the walking or weight bearing position and supine.

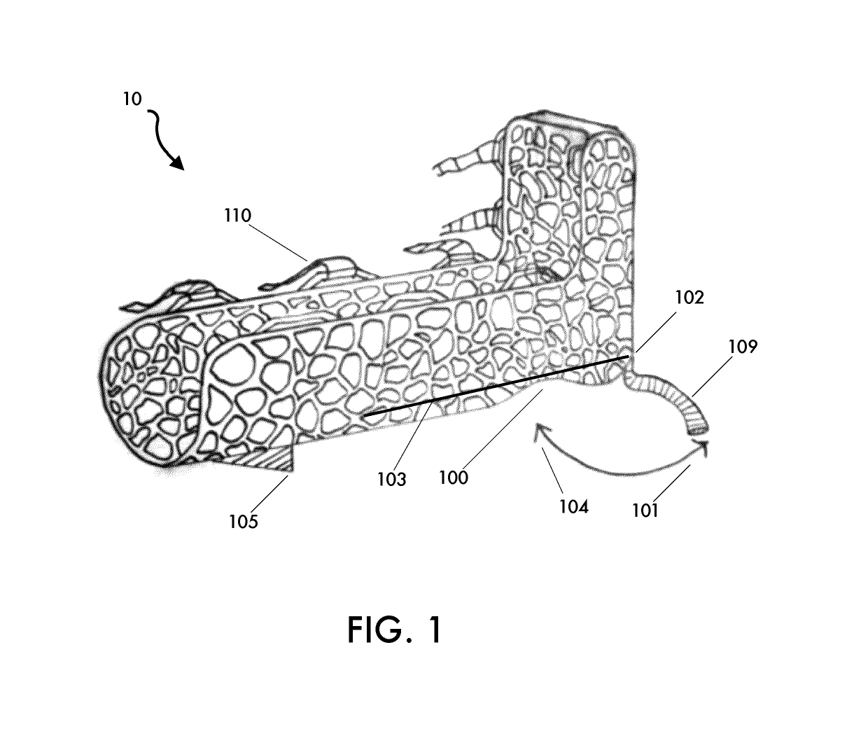

Now turning to FIG. 1, a detailed side perspective of the orthopaedic brace 10 is shown, showing the rigid, generally L-shaped exoskeleton having an elongate, open-topped channel dimensioned to receive and seat a lower leg and foot of a patient. According to an embodiment of this invention, the lower leg while seated or laying down within the rigid exoskeleton of the orthopaedic brace 10 is aligned with a major axis 103 of the rigid exoskeleton. Further to this embodiment, stabilizer structures 105 associated with the rigid exoskeleton of the orthopaedic brace 10, are dimensioned to interface with a generally horizontal support surface to, while being supported, inhibit rotation of the rigid exoskeleton about the major axis 103. According to an aspect of this embodiment, at least one stabilizer plate 105 depending from a side of the rigid exoskeleton of the orthopaedic brace 10 opposite the open-mouth of the channel and terminating at a distal edge that runs generally transverse to the major axis 103. These may extend from opposite sides of the open mouth of the channel of the rigid exoskeleton. According to another embodiment, the extendable stabilizers 102, 109 has an upper portion extending generally along the rigid exoskeleton 102 and a lower portion extending generally away from the rigid exoskeleton 109. According to an aspect of this embodiment, the lower portion 109 is selectively bendable and thinner with respect to the upper portion 102 thereby to permit selective adjustment. Each of the stabilizer legs is selectively movable and pivotable between a retracted position 104 and a stabilizing position 101.

According to another embodiment, the rigid exoskeleton that conforms to at least a major portion of the lower leg and foot may be a monocoque structure, or two or more connectable members. Any variation of sizes in these two separate pieces may be allowed such that a proper fit is achieved.

According to an aspect of this embodiment, at least one cushion member associated with the rigid exoskeleton of the orthopaedic brace 10 for cushioning a lower leg and foot while seated within the rigid exoskeleton may be used. This cushion may be comprised of foam, be removable and/or be integral with the rigid exoskeleton of the orthopaedic brace 10. It may further be comprised of a Voronoi-patterned structure.

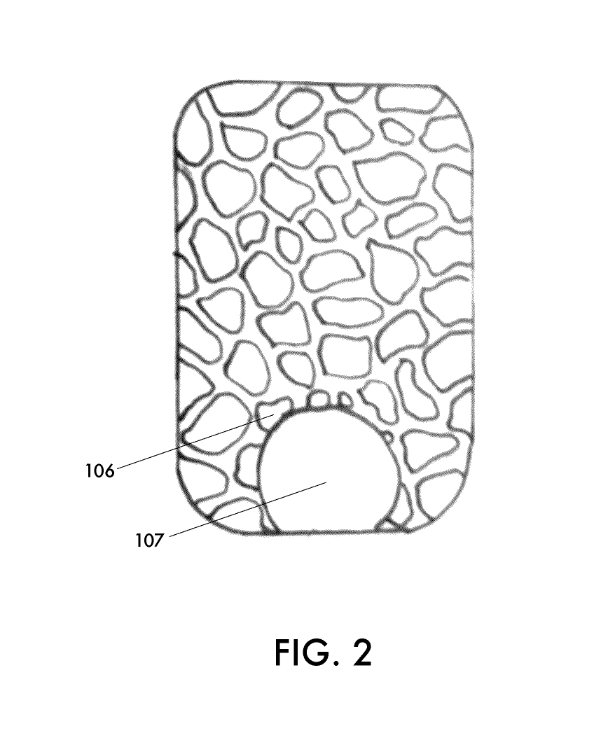

As shown particularly in FIG. 2, the end-on view of the planter portion of the orthopaedic brace exoskeleton 10 is visible. In this embodiment, the variations in the voronoi pattern is visible 106 where the weight barring portion of the heel makes it necessary for a tighter pattern of veins, and where the heel makes direct contact and a solid surface may be required 107 with cushioning.

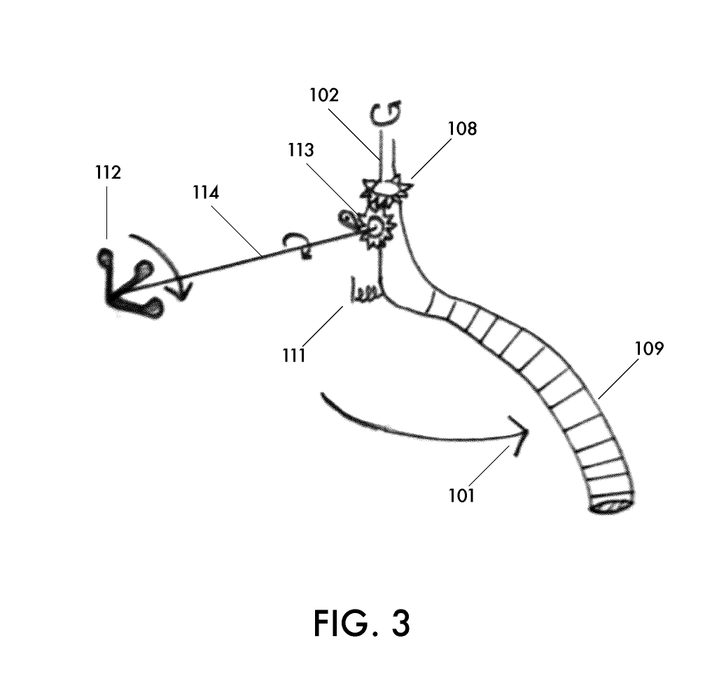

As shown particularly in FIG. 3, a side perspective of the stabilizers 102, 109 extendable through a spring loaded ratcheting mechanism is visible. In this embodiment, the outer portion of the stabilizer 109 that prevents the leg from medial or lateral rotation and extends through a spring-loaded 111 ratcheting mechanism 113 is selectively movable and pivotable between a retracted position 104 and a stabilizing position 101. Each of the stabilizer legs 102, 109 is connected to the rigid exoskeleton of the orthopaedic brace 10 in the region of 102, thereby enabling the selective pivoting. Here, each spring-loaded 111 ratcheting mechanism 113 biases the respective stabilizer leg to the retracted position 104 through an inlet 100 for receiving the stabilizer legs. According to an aspect of this embodiment, extension the stabilizers 102, 109 to the stabilizing position 101, may be done by a user-operable control, selected from the group consisting of a hand-operated lever, a crank, and a knob 112. Through the use of these devices, the stabilizers 102, 109 are torqued into the position roughly perpendicular from the length lateral to the leg 101 and relaxed in the position lateral to the leg through a rod 114 and gear mechanism 108.

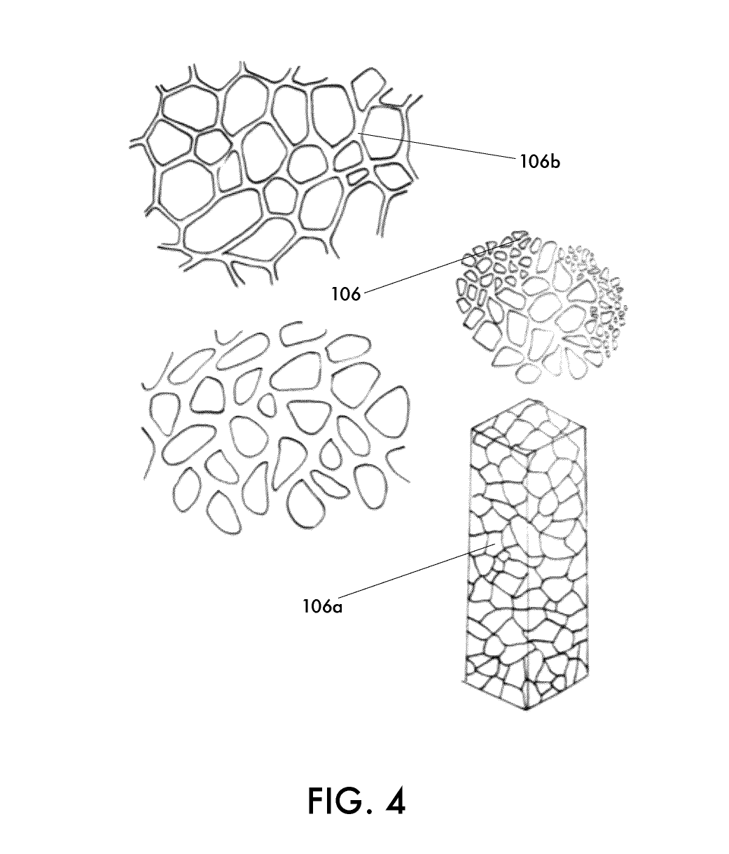

As shown particularly in FIG. 4, there is shown several examples, though not limiting, of the Voronoi pattern 106 which could be used, including a view of the pattern as it fills the 3D space of an elongated rectangle 106a. In this embodiment, the voronoi patterns shown contain a variety of organic shapes inspired by cellular clustering patterns in the control of stiffness variation. Stiffness variation corresponds to multi-scalar loading conditions and the density of the various organic shapes. The relative thickness of the vein-like elements 106b modelling the surface relates to pressure and stress differentiations.

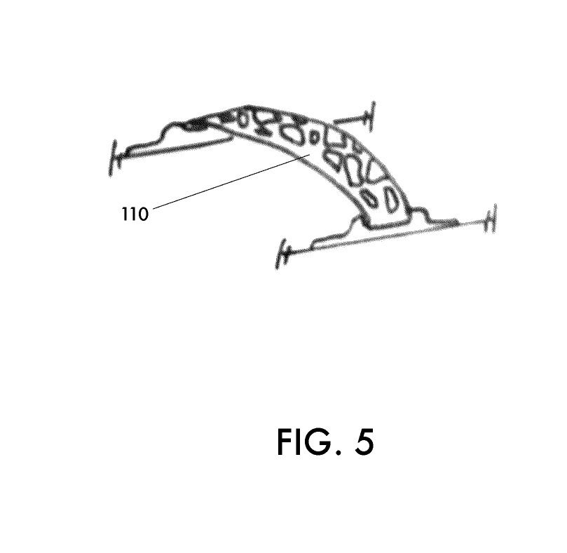

As shown particularly in FIG. 5, there is shown a side perspective of the retention device 110 showing the Voronoi pattern 106. According to this embodiment, at least one retention device 110 associated with the rigid exoskeleton of the orthopaedic brace 10 would be included, and may be adjustable or contain a Voronoi-patterned structure 106. According to an aspect of this embodiment, the pattern may apply equally well to the padding of the strap, as well as to the structural portion of the strap.

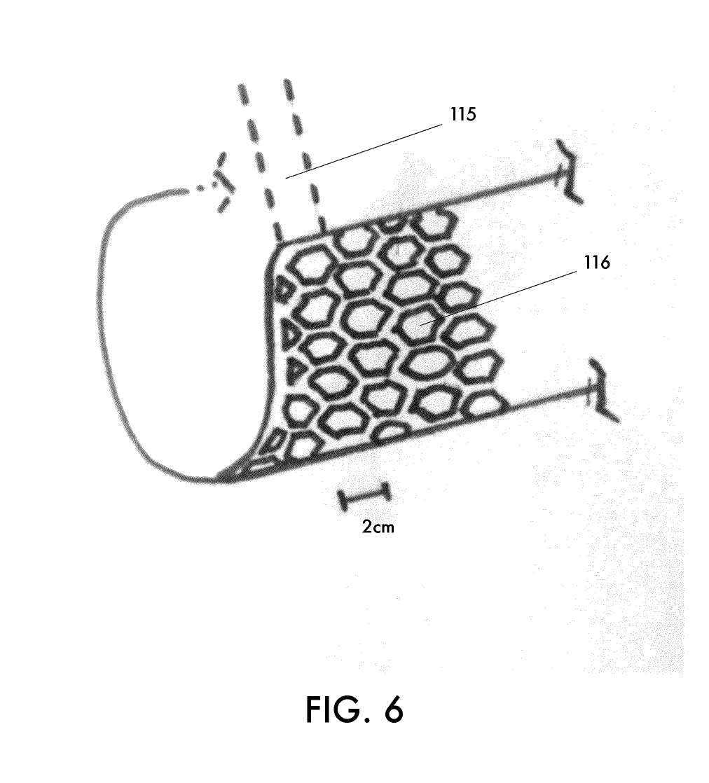

As shown particularly in FIG. 6, the side perspective of the rigid exoskeleton of the orthopaedic brace 10 is shown. In this embodiment, the design of the rigid exoskeleton may contain a hexagonal-patterned structure 116, wherein the hexagonal-patterned structure 116 comprises hexagonal cells each having a diameter of from about 1 centimeter to about 2 centimeters 115, and can be formed from fibreglass impregnated resin and/or any plastic or polymer suitable for extracorporeal medical devices and/or additive manufacturing and may have a smooth surface. These materials may also be used in the rigid exoskeleton comprising a Voronoi-patterned structure.

According to another embodiment of this invention, a method for producing a three-dimensional model is given, comprising conducting a three-dimensional scan of the lower leg and foot of the patient either before or after a surgery to produce a computer-generated file, which may be printed using additive manufacturing.

The above-described improvements to the orthopaedic ankle brace can also be applied to knee braces. Such improvements may apply equally well, mutatis mutandis, with such mutations as being relevant, including but not limited to, knee braces, wrist braces, neck braces, back braces, and other things.

Some embodiments may have been described with reference to method type claims whereas other embodiments may have been described with reference to apparatus type claims. However, a person skilled in the art will gather from the above and the following description that, unless other notified, in addition to any combination of features belonging to one type of subject matter also any combination between features relating to different subject matters, in particular between features of the method type claims and features of the apparatus type claims is considered as to be disclosed with this document.

The aspects defined above and further aspects are apparent from the examples of embodiment to be described hereinafter and are explained with reference to the examples of embodiment.

Although embodiments have been described with reference to the drawings, those of skill in the art will appreciate that variations and modifications may be made without departing from the spirit and scope thereof as defined by the appended claims.

1. An orthopaedic brace comprising:
a rigid, generally L-shaped exoskeleton having an elongate, open-topped channel dimensioned to receive and seat a lower leg and foot of a patient, wherein the lower leg while seated within the rigid exoskeleton is aligned with a major axis of the rigid exoskeleton;
stabilizer structure associated with the rigid exoskeleton, the stabilizer structure dimensioned to interface with a generally horizontal support surface to, while being supported, inhibit rotation of the rigid exoskeleton about the major axis.

2. The orthopaedic brace of claim 1, further comprising:
at least one retention device associated with the rigid exoskeleton for retaining a lower leg and foot while seated within the rigid exoskeleton.

3. The orthopaedic brace of claim 2, wherein each of the at least one retention device comprises an adjustable strap.

4. The orthopaedic brace of claim 2, wherein each of the at least one retention device comprises a Voronoi-patterned structure.

5. The orthopaedic brace of claim 1, further comprising:
at least one cushion member associated with the rigid exoskeleton for cushioning a lower leg and foot while seated within the rigid exoskeleton.

6. The orthopaedic brace of claim 5, wherein the at least one cushion member is comprised of foam.

7. The orthopaedic brace of claim 5, wherein the at least one cushion member is integral with the rigid exoskeleton.

8. The orthopaedic brace of claim 5, wherein the at least one cushion member is removable from the rigid exoskeleton.

9. The orthopaedic brace of claim 5, wherein the at least one cushion member comprises a Voronoi-patterned structure.

10. The orthopaedic brace of claim 1, wherein the rigid exoskeleton is comprised of resin impregnated with fiberglass.

11. The orthopaedic brace of claim 1, wherein the rigid exoskeleton comprises a Voronoi-patterned structure.

12. The orthopaedic brace of claim 1, wherein the rigid exoskeleton comprises a smooth exterior surface.

13. The orthopaedic brace of claim 1, wherein the rigid exoskeleton comprises a hexagonal-patterned structure.

14. The orthopaedic brace of claim 13, wherein the hexagonal-patterned structure comprises hexagonal cells each having a diameter of from about 1 centimeter to about 2 centimeters.

15. (canceled)

16. (canceled)

17. The orthopaedic brace of claim 1, wherein the stabilizer structure comprises:
at least one stabilizer plate depending from a side of the rigid exoskeleton opposite the open-mouth of the channel and terminating at a distal edge that runs generally transverse to the major axis.

18. The orthopaedic brace of claim 1, wherein the stabilizer structure comprises:
first and second stabilizer legs extending from opposite sides of the open mouth of the channel of the rigid exoskeleton.

19. The orthopaedic brace of claim 18, wherein each of the first and second stabilizer legs has an upper portion extending generally along the rigid exoskeleton and a lower portion extending generally away from the rigid exoskeleton.

20. The orthopaedic brace of claim 19, wherein the lower portion is selectively bendable with respect to the upper portion thereby to permit selective adjustment.

21. (canceled)

22. The orthopaedic brace of claim 18, wherein each of the stabilizer legs is selectively movable between a retracted position and a stabilizing position.

23. (canceled)

24. (canceled)

25. (canceled)

26. (canceled)

27. (canceled

28. (canceled)

29. A method for stabilizing a patient's hip, the method comprising:
sizing the lower leg and foot of the patient;
constructing the orthopaedic brace of claim 1 comprising forming the rigid exoskeleton according to the sizing; and
causing the orthopaedic brace to receive and seat the lower leg and foot of the patient,
wherein the generally-horizontal support surface is a patient bed.

30. (canceled)

31. (canceled)

32. (canceled)

33. (canceled)
